(19) United States
(12) Patent Application Publication
(10) Pub. No.: US 2023/0140245 A1
(43) Pub. Date: May 4, 2023

(54) DEVICES, SYSTEMS, AND METHODS FOR CONTROLLING AND VARYING MEDICAL INSTRUMENT MONITORING OF PATIENTS AND FOR SWITCHING BETWEEN SYNCHRONOUS AND ASYNCHRONOUS MONITORING

(71) Applicant: Darroch Medical Solutions, Inc., San Diego, CA (US)

(72) Inventors: Subramanian Shastri, Carlsbad, CA (US); Anthony Shao, Hillsborough, CA (US); Colby Bishop, San Diego, CA (US); An Tran, San Diego, CA (US)

(21) Appl. No.: 17/979,797

(22) Filed: Nov. 3, 2022

Related U.S. Application Data
(60) Provisional application No. 63/275,639, filed on Nov. 4, 2021.

(51) Int. Cl. A61B 5/00; A61M 5/168; G01G 19/44; G01G 19/52

(52) U.S. Cl. CPC A61B 5/7275; A61B 5/746; A61M 5/16854; A61B 5/6892; G01G 19/445; G01G 19/52; A61B 2560/045; A61M 2205/18

(57) ABSTRACT

A communication system for controlling and varying medical instrument monitoring of patients and switching between synchronous and asynchronous monitoring is provided comprising a data analytics system and a dongle including a microcontroller and an antenna in communication with the microcontroller. The dongle is configured to receive data from a medical instrument, and the data includes one more metrics being measured by the medical instrument. The dongle is configured to send periodic signals of a first frequency to the medical instrument. When one or more of the metrics exceeds a pre-determined boundary parameter or one or more aggregated metrics generate a high patient risk score, the dongle increases the frequency of the periodic signals. The communication system can route a signal via a synchronous input/output unit to the medical instrument to cause the medical instrument to provide regular readings of the data and to send a signal via an asynchronous input/output unit to the medical instrument to cause the medical instrument to provide additional or irregular readings of the data and to switch between the synchronous input/output unit and the asynchronous input/output unit on demand or based on prior instructions from a user.

### CROSS-REFERENCE TO RELATED APPLICATION

This application is a non-provisional of and claims priority to U.S. Patent Application 63/275,639, filed Nov. 4, 2021, which is hereby incorporated by reference in its entirety.

### FIELD OF THE DISCLOSURE

The present disclosure relates to devices, systems, and methods for controlling and signaling medical instruments. More particularly, the present disclosure relates to proprietary dongles and data analytics systems for varying medical instrument monitoring of patients and for switching back and forth between synchronous and asynchronous monitoring.

### BACKGROUND

Adverse events (AEs) in hospitalized patients are defined as “unintended injuries or complications that result in disability at discharge, death, or prolonged hospital stay and are caused by events other than the patient's underlying disease.” Up until recently, adverse events were assumed to be sudden, unpredictable, and difficult to avoid. However, recent institutional studies have shown that many common adverse events have established precursors that often come in the form of detectable and anomalous variations in vital signs, or other medical instrument metrics, hours or even days before their onset. Unfortunately, due to limitations in technology, human resources, and finance, many of these adverse event precursors go unnoticed and become harmful or fatal.

There are two primary limitations in the current healthcare monitoring ecosystem for realizing AE markers. The first limitation can be attributed to the lack of “real time” data output from many treatment and monitoring devices. Currently, patient medical instrument information is either manually transcribed by the attending staff or is batch-processed into the Electronic Medical Records (EMRs) over expensive proprietary servers. In many cases, this batch-processing occurs at specific hourly intervals (e.g. every 2, 4, 6 hours). Patients who deteriorate between these specified intervals may not receive the increased treatment required, and practitioners who rely on the antiquated data sets may form an incorrect interpretation of the patient's current physiological status, leading to incorrect knowledge and treatment.

The second limitation can be attributed to the demanding patient-to-nurse ratios in non-intensive care units (non-ICUs); nurses may be required to monitor up to six patients in these environments. Because healthcare practitioners must manually assess the status of each patient's instruments during each round to ascertain the patient's physiological status, it can be almost impossible to ascertain each patient's physiological status, determine if AEs are to occur, and generate clinical protocols. Thus, if a patient deteriorates in between the nurse rounds, he or she may not receive the appropriate medical attention required. In addition, nurse experience can play a vital role in proper judgment and action for potentially deteriorating patients, and it can be difficult to internalize individual patient metrics and determine protocol for several patients at once.

Though many Early Warning Systems (EWS) and AE prevention systems have been developed in recent years, many tend to fall short in practical applications for three main reasons. The first shortcoming of these systems is that different health metrics change at different speeds. For example, heart rate and blood oxygen can change relatively quickly over the course of fifteen minutes. On the other hand, temperature is a much slower variable that may require a much longer timeframe to see changes over time.

A second limitation of current EWS is the varying time scales at which data is measured, as many of the measured metrics may have asynchronous time scales for measurement. For example, heart rate may be measured every second while other metrics, such as blood pressure or temperature, may be taken manually every fifteen minutes.

A third shortcoming of current early EWS is a derivative of the varying health metric speeds and asynchronous timescales, as the difference between the retroactive data sets used to train the predictive models and actual, live patient data can vary substantially. Models that are trained using retroactive data sets are fed a consistent set of data at specified intervals; this is to optimize the potential for the most accurate anticipatory diagnosis.

However, in many live environments, the data sets that are fed into the EWS are incongruent to the original trained data sets; this can be for a variety of reasons—manually inputted metrics may not have been taken, batch-processed EMR data comes late, or the asynchronous time scales create “lag” between the specified time analysis intervals (e.g., current heart rate data may be analyzed with antiquated blood pressure data). In addition, the data sets required to train “real time”, low latency AE models can be incredibly difficult to find. In the current healthcare ecosystem, many AE models are trained on EMR data, which as discussed, often do not have high frequency data.

Thus, there is a substantial need for a system that can determine the status of the patient, normalize measurement frequencies, and relay relevant data to attending practitioners for protocol execution on a multi-patient front.

### SUMMARY

The present disclosure, in its many embodiments, alleviates to a great extent the disadvantages of known EWS and AE prevention systems by providing a communication system for controlling and varying medical instrument monitoring and switching between synchronous and asynchronous monitoring. Exemplary systems include a proprietary dongle and/or a proprietary communication device. The device, system and dongle vary signal frequencies and data aggregation frequencies based on adverse event potential, such as event-based precursors or patient risk scores. The higher the system determines a patient's adverse event potential, the more regular and frequent signaling and data measurements become. When a measured metric exceeds a boundary or aggregated metrics generate a high patient risk score, the dongle increases the frequency of periodic signals to the medical instrument, and the system sends an alarm to a medical practitioner.

The dongle and/or communication device may have a synchronous input/output unit and an asynchronous input/output unit. The dongle and/or communication device may be configured to route the signals to the synchronous input/output unit to cause the medical instrument to provide regular readings of the data and to the asynchronous input/output unit to cause the medical instrument to provide additional or irregular readings of the data and to switch between the synchronous input/output unit and asynchronous input/output unit on demand or based on instructions from a user. Disclosed embodiments improve the functioning and utility of medical instruments such as infusion pumps, vital-signs monitors, and bed scales, making them more useful to medical practitioners.

An exemplary dongle configured to communicate with a medical instrument comprises a microcontroller and an antenna in communication with the microcontroller and may also have an IUI connector. The dongle is configured to receive data from a medical instrument, and the data include one or more metrics being measured by the medical instrument. The dongle is configured to send periodic signals of a first frequency to the medical instrument. When one or more of the metrics exceeds a pre-determined boundary parameter or one or more aggregated metrics generate a high patient risk score, the dongle sends periodic signals of a second frequency which is higher than the first frequency to the medical instrument. In exemplary embodiments, when a single metric exceeds the pre-determined boundary parameter the second frequency is a medium frequency, and when more than one metric exceeds the pre-determined boundary parameter the second frequency is a high frequency.

When more than one metric exceeds the pre-determined boundary parameter the dongle may prompt the medical instrument to manually measure the more than one metric at specified time intervals. In exemplary embodiments, changes in frequency of the signals sent by the dongle are enabled by hardware changes comprising changes in component voltages or changes in crystal-based frequencies. In exemplary embodiments, the microcontroller compares the measured metrics to the pre-determined boundary parameters. The microcontroller analyzes aggregated measured metrics for adverse event anticipation and to generate the patient risk score and uses the patient risk score to normalize asynchronous specified time intervals for more than one measured metric.

A communication system for controlling and varying medical instrument monitoring comprises a dongle including a microcontroller and an antenna in communication with the microcontroller, and a data analytics system including a computer-readable storage medium and implementing instructions stored in the computer-readable storage medium. The dongle is configured to receive data from a medical instrument, and the data includes one more metrics being measured by the medical instrument. The dongle is configured to send periodic signals of a first frequency to the medical instrument. When one or more of the metrics exceeds a pre-determined boundary parameter or one or more aggregated metrics generate a high patient risk score, the dongle sends periodic signals of a second, higher, frequency to the medical instrument.

Changes in frequency of the signals sent by the dongle are enabled by the instructions stored in the computer-readable storage medium. When a single metric exceeds the pre-determined boundary parameter the second frequency is a medium frequency, and when more than one metric exceeds the pre-determined boundary parameter the second frequency is a high frequency. In exemplary embodiments, when more than one metric exceeds the pre-determined boundary parameter the dongle prompts the medical instrument to manually measure the more than one metric at specified time intervals.

In exemplary embodiments, the data analytics system analyzes aggregated measured metrics for adverse event anticipation to generate the patient risk score and uses the patient risk score to normalize asynchronous specified time intervals for more than one measured metric. The data analytics system may determine whether any of the data from the medical instrument is outside the pre-determined boundary parameter and determines likelihoods that a patient may experience an adverse event through comparisons between the data from the medical instrument and data in an adverse event repository.

The communication system may further comprise an early warning system including a user interface and an alarm. The communication system communicates information about a patient's medical condition based on the data from the medical instrument to a mobile device in real time. In exemplary embodiments, the user interface displays the results of the comparisons between the data from the medical instrument and the data in the adverse event repository. The alarm alerts the medical practitioner when stored data is outside a pre-determined boundary parameter, or the medical instrument has stopped functioning or changed its rate, or a patient may experience an adverse event.

The medical instrument may be an infusion pump, a vital-signs monitor, and/or a bed scale. In cases where the medical instrument is an infusion pump, the alarm may alert a medical practitioner when pressure builds on an IV line connected to the infusion pump. When the medical instrument is a bed scale including a load sensor, the dongle collects load scale data from the load sensor and the data analytics system analyzes the load scale data and presents the analyzed load scale data on the user interface.

An exemplary embodiment of a communication system for switching between synchronous and asynchronous monitoring of a patient comprises a dongle including a microcontroller, a communication device in communication with the dongle, a synchronous input/output unit, an asynchronous input/output unit, and a data analytics system. The data analytics system includes a computer-readable storage medium and implementing instructions stored in the computer-readable storage medium. The dongle is configured to receive data from a medical instrument, and the data includes one more metrics being measured by the medical instrument.

The communication system is configured to route a signal via the synchronous input/output unit to the medical instrument to cause the medical instrument to provide regular readings of the data and to route a signal via the asynchronous input/output unit to the medical instrument to cause the medical instrument to provide additional or irregular readings of the data and to switch between the synchronous input/output unit and the asynchronous input/output unit on demand or based on prior instructions from a user. In exemplary embodiments, the dongle has the synchronous input/output unit and the asynchronous input/output unit. In exemplary embodiments, the communication device has the synchronous input/output unit and the asynchronous input/output unit.

Accordingly, it is seen that devices, systems, and methods of controlling and varying medical instrument monitoring of patients and switching between synchronous and asynchronous monitoring are provided. These and other features and advantages will be appreciated from review of the following detailed description, along with the accompanying figures in which like reference numbers refer to like parts throughout.

### BRIEF DESCRIPTION OF THE DRAWINGS

The above-mentioned features and objects of the present disclosure will become more apparent with reference to the following description taken in conjunction with the accompanying drawings wherein like reference numerals denote like elements and in which:

FIG. 1 is a diagram of an exemplary embodiment of a system for controlling and varying medical instrument monitoring in accordance with the present disclosure;

FIG. 2A is a bottom view of an exemplary embodiment of a dongle in accordance with the present disclosure;

FIG. 2B is a top view of an exemplary embodiment of a dongle in accordance with the present disclosure;

FIG. 2C is a top view of an exemplary embodiment of a dongle in accordance with the present disclosure;

FIG. 3 is a front view of an exemplary embodiment of an early warning system showing an exemplary home screen in accordance with the present disclosure;

FIG. 4 is a schematic of an exemplary embodiment of a dongle adjusting signal frequency in accordance with the present disclosure;

FIG. 5A is a front view of an exemplary embodiment of a system for controlling and varying medical instrument monitoring in accordance with the present disclosure showing an exemplary patient risk score;

FIG. 5B is a front view of an exemplary embodiment of a system for controlling and varying medical instrument monitoring in accordance with the present disclosure showing an exemplary patient risk score;

FIG. 6 is a flow chart of an exemplary embodiment of a method controlling and varying medical instrument monitoring in accordance with the present disclosure showing normalization of asynchronous time scales;

FIG. 7 is a front view of an exemplary embodiment of a user interface showing an exemplary home screen in accordance with the present disclosure;

FIG. 8 is a front view of an exemplary embodiment of a user interface showing an exemplary in-depth screen in accordance with the present disclosure;

FIG. 9 is a front view of an exemplary embodiment of a user interface showing an exemplary graphing screen in accordance with the present disclosure;

FIG. 10 is a flow chart of an exemplary method of controlling and varying medical instrument monitoring in accordance with the present disclosure;

FIG. 11 is a flow chart of an exemplary method of controlling and varying medical instrument monitoring including switching between synchronous and asynchronous monitoring and pushing different data frequencies in accordance with the present disclosure; and

FIG. 12 is a diagram of an exemplary system of controlling and varying medical instrument monitoring including switching between synchronous and asynchronous monitoring and pushing different data frequencies in accordance with the present disclosure.

### DETAILED DESCRIPTION

In the following detailed description of exemplary embodiments of the disclosure, reference is made to the accompanying drawings in which like references indicate similar elements, and in which is shown by way of illustration specific embodiments in which disclosed devices, systems, and methods may be practiced. These embodiments are described in sufficient detail to enable those skilled in the art to practice the embodiments, and it is to be understood that other embodiments may be utilized, and that logical, mechanical, functional, and other changes may be made without departing from the scope of the present disclosure. The following detailed description is, therefore, not to be taken in a limiting sense, and the scope of the present disclosure is defined by the appended claims. As used in the present disclosure, the term “or” shall be understood to be defined as a logical disjunction and shall not indicate an exclusive disjunction.

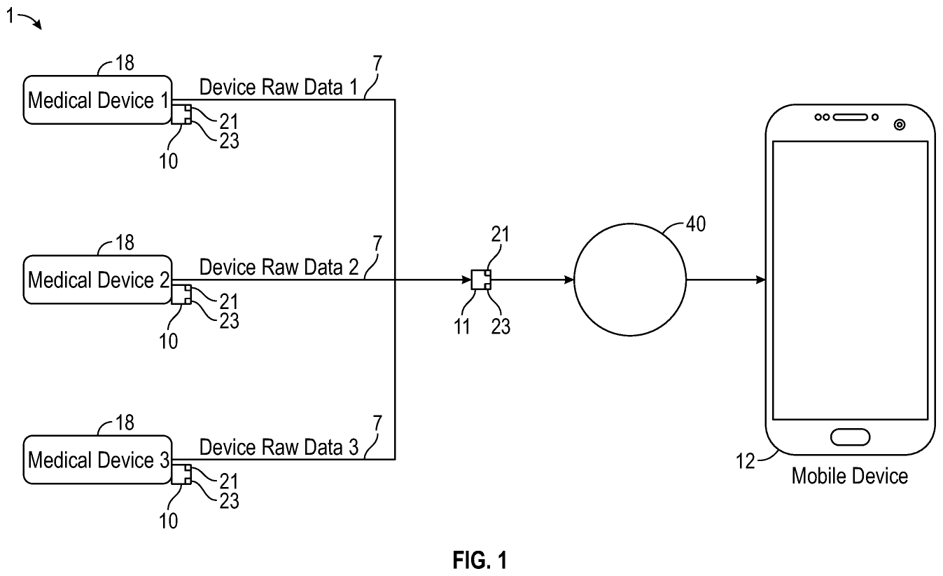

FIG. 1 illustrates an exemplary embodiment of a system 1 for controlling and varying medical instrument monitoring of patients and for switching between synchronous and asynchronous monitoring. The system 1 communicates with medical instruments or devices 18 and can actively vary the aggregation and transmission rates of medical data 7 from the instruments 18. The variable data communication system 1 includes a dongle 10, which may be any kind of adapter or other device that can plug into or otherwise communicatively couple to a computer or medical instrument 18 and transmit and receive signals between the medical instruments 18 and mobile devices 12 and collect and aggregate medical instrument data 7.

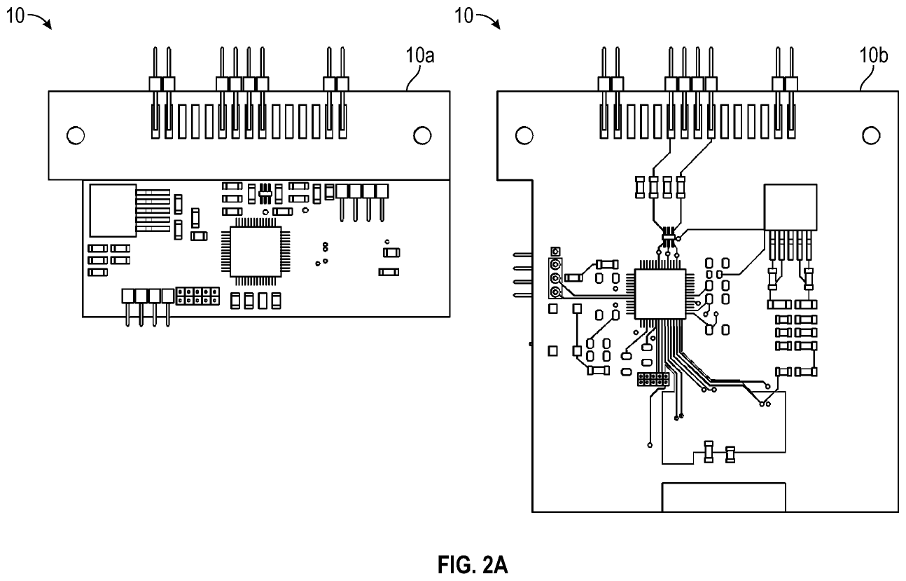

In exemplary embodiments, as shown in FIGS. 2A, 2B and 2C, a proprietary dongle 10 (shown in two possible sizes, 10a and 10b) is created through a normal small microcontroller 14 (e.g., microchip SAMD21A1 board), antenna 16, and optionally an IUI connector (not shown) when used with an infusion pump. As best seen in FIG. 1, the dongle 10 may have a synchronous input/output (I/O) unit 21 and an asynchronous I/O unit 23 and be configured to route through either.

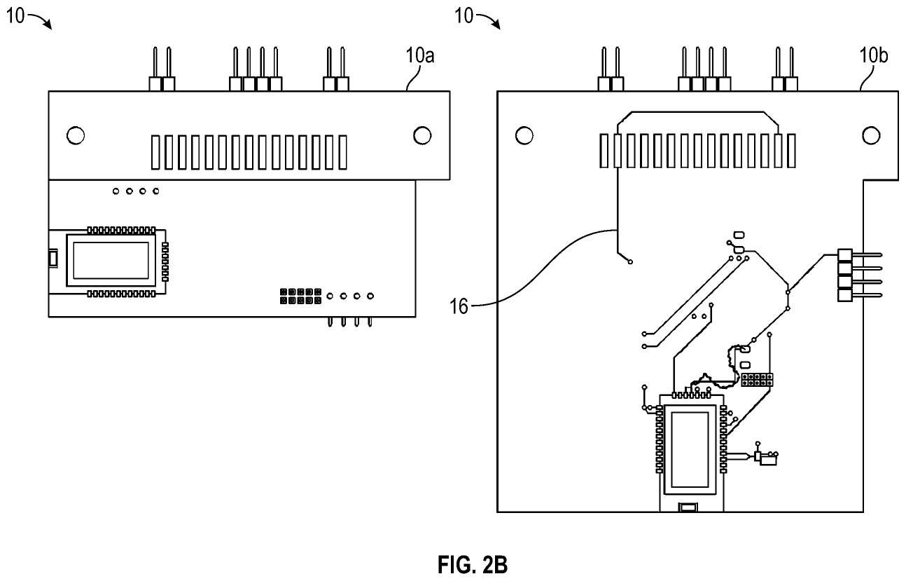

As described in more detail herein, the communication system 1 includes a data analytics system 40 for performing various analytical functions on the data collected from the medical instruments 18. It has a computer-readable storage medium with implementing instructions for implementing its various functions. A communication device 11 also may be provided to receive and coordinate data from various medical instruments and present it in a cohesive manner to any medical practitioner qualified to view it. The communication device may have a synchronous I/O unit 21 and an asynchronous I/O unit 23 and be configured to route through either. As shown in FIG. 3, an early warning system 22 is also provided as party of the communication system 1 and typically includes a user interface 30 and an alarm 35, which may be visual, auditory, and/or vibratory.

Disclosed embodiments can work in conjunction with or as part of communication systems, devices, patient proxies, and related methods described in U.S. patent application Ser. No. 15/962,290, filed Apr. 25, 2018, co-pending U.S. patent application Ser. No. 16/594,111, filed Oct. 7, 2019, and U.S. patent application Ser. No. 16/784,727, filed Feb. 7, 2020, each of which is hereby incorporated by reference in its entirety. Data analytics systems and methodology described in the above-referenced patent applications provide nurses and other medical practitioners with real-time remote monitoring and analysis of their patient's treatment and monitoring devices (e.g., vital signs monitors, IV pumps, and bed scales). This information can be viewed on mobile devices 18 to help anticipate potential AE markers via the dongles 10.

In exemplary embodiments, the medical instruments 18 the system 1 communicates with are an infusion pump, a vital signs monitor, and/or a bed scale. The metrics in the medical data 7 measured and communicated depends of course on the different medical instruments. The metrics measured by the vital-signs monitors include, but are not limited to, heart rate (pulse), respiration rate, blood pressure (systolic and diastolic), temperature, and oxygen saturation. For the infusion pumps, the measured metrics include, but are not limited to, volume to be infused (VTBI), set flow rate, actual flow rate, pressure in line, pressure exerted from the vein, time left in infusion. The bed scales measure patient activity as a result of variations in the load cells within the bed that measure the patient's weight.

It should be noted that exemplary embodiments advantageously provide certain medical data and metrics to the user that are not traditionally collected by the medical instruments 18. For example, traditional bed scales do not collect and communicate load scale data. But embodiments of the specialized dongle 10 can be appended to certain beds and allow for collection of this type of information (and subsequently presentation). The data analytics system 40 interprets load sensor measurements, sums the voltages together, and presents the output on the user's mobile device, which the nurse can use to determine if the patient is restless. In addition, the infusion pump dongle can retrieve data from an infusion pump that is not displayed on the infusion pump itself, such as when pressure builds on an IV line connected to the pump.

The aggregated metrics are used by the system 1 in various ways. They may be used to create patient-specific profiles (“patient proxies”) that allow nurses to understand patient physiology without having to physically assess the patient's medical instruments. The data analytics system 40 also allows users to set alarm conditions when certain criteria are met. For example, users can set boundary conditions on any of the measured metrics to be alerted when a set of data points is outside of the specified conditions. Finally, the medical data 7 aggregated by the data analytics system 40 is used as training data sets for AE anticipation.

Advantageously, the proprietary dongle 10 can adjust signal frequency to actively vary the collection, aggregation, and transmission rates of medical data 7 from the medical instruments 18. The dongle 10 can do this independently or as incorporated into the full system 1. This is critically important because patients who deteriorate in between nurse rounds or EMR collection times may have a higher chance of experiencing an AE. In addition, not all patients require the highest level of data aggregation, and power and communication constraints can hamper the system's 1 ability to relay aggregated metrics at the highest frequency. Operating at the highest data aggregation frequencies would be resource intensive and could cause systems to fail, so it is advantageous that disclosed dongles change to a fast aggregation architecture only when needed.

Therefore, disclosed communication systems 1 can vary medical instrument monitoring frequencies based on adverse event potential, such as event-based precursors or patient risk scores. The higher the system determines a patient's adverse event potential is, the more regular and frequent data measurements become. These processes can be a mix of statistical manipulation, derivations to determine changes over time, and more.

The communication system 1 utilizes multiple methodologies for varying the rate of medical data 7 collection. In the case of event-based precursors, the system 1 can increase the signal frequency from the dongle 10 or communication device 11 to the medical instrument 18, causing the medical instrument to increase its monitoring rate. One such example is a comparison between the metric measurements aggregated and their relation to the boundary conditions set by the user.

Exemplary embodiments allow the user to set boundary conditions 33 for many of the metrics measured by the medical instruments 18. For example, the user may set the data analytics system 40 to alert the user when the patient's heart rate is not in between the values of 60-130 bpm or the pulse is lower than 54 or higher than 137. These boundary conditions are primarily designed to alert the user when a data measurement is outside of the specified conditions and are conjunctively used to determine whether a patient is exhibiting adverse event precursors. Patients who exhibit multiple metrics that breach a boundary are often at a higher risk of developing an AE and thus require a higher level of monitoring. The data analytics system 40 also determines whether any of the medical instrument data is outside the pre-determined boundary parameter and determines likelihoods that a patient may experience an adverse event through comparisons between the data from the medical instrument and data in an adverse event repository.

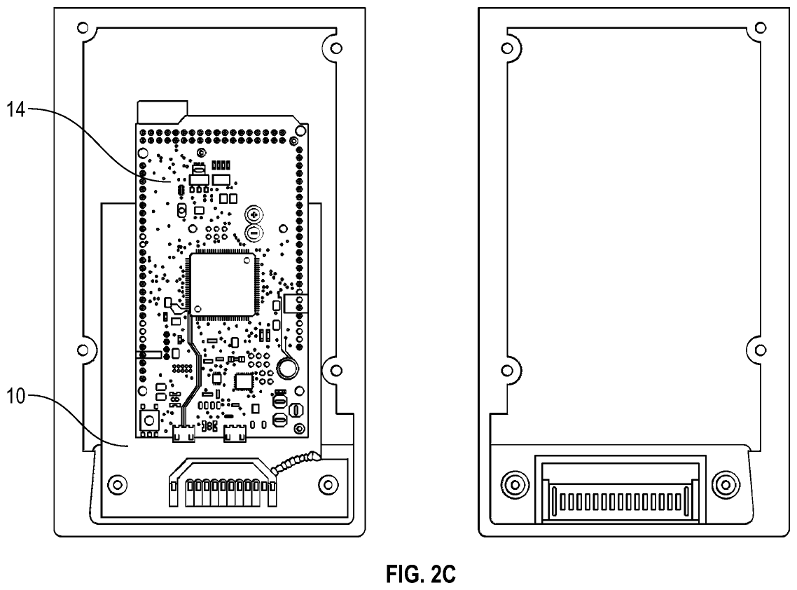

As illustrated in FIG. 4, the dongle 10 is configured to send periodic signals to a medical instrument 18 at a baseline or default frequency. In exemplary embodiments, if a single metric is breached, exceeding a pre-determined boundary parameter for a prolonged period, the system 1 increases its frequency of measurement from a default state of low frequency to medium frequency. Thus, the dongle 10 will ping the medical instrument 18 starting at first low frequency 15, then at a second, higher frequency 17, e.g., 1 sample/min=>2 samples/min, or any suitable increase so the patient is more frequently monitored for the metric.

In exemplary embodiments, the baseline (low) frequency of data aggregation is somewhere along the lines of one sample every ten minutes, the medium rate of data aggregation is an order of five higher, that is one sample every two minutes, and the high frequency data aggregation is four times higher at one sample every thirty seconds, but these rates could vary depending on the metric, the medical practitioners, and the institution. It should be noted that in certain embodiments, communication device 11 may be configured to send signal to the medical instruments 18, and either the dongle 10 or the communication device 11 can perform all the signaling functions described herein.

If more than one metric is breached, exceeding a pre-determined boundary parameter for a prolonged period, the system 1 increases its frequency of measurement from the existing state of low 41 or medium frequency 43 to high frequency 45 in response to dongle 10 or communication device 11 signals of low 15, medium 17, and high frequency 19. Thus, the dongle 10 will increase the pinging speed to a high frequency if more than one metric is breached. In exemplary embodiments, the dongle 10 may also begin prompting the medical instrument 18 for manually measured metrics at specified time intervals that are consistent with higher yielding adverse event anticipation systems. The default settings for low, medium, and high frequency typically would be consistent with the medical institution's protocol.

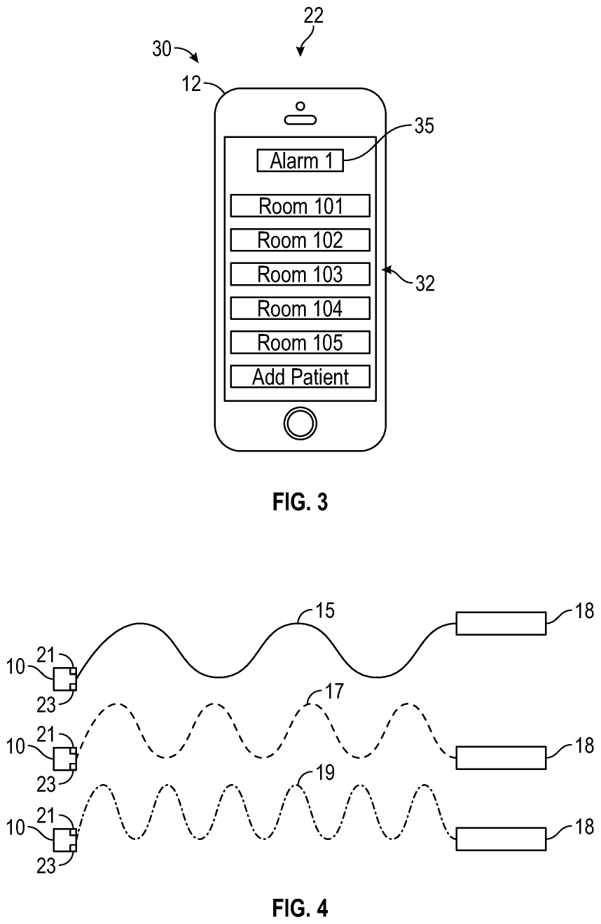

With reference to FIGS. 5A and 5B, another method of varying the rate of medical data 7 collection is to do so based on a “patient risk score.” This is necessary because some patients may have AE precursors without creating a breach in boundary conditions. For this reason, the data analytics system 40 advantageously calculates a patient risk score 38. More particularly, the data analytics system analyzes aggregated measured metrics in the medical data for adverse event anticipation to generate a patient risk score 38. The metrics may be analyzed as a group for either specific event anticipation (e.g., sepsis anticipation through the SIRS criteria) or generalized adverse event anticipation (e.g., use of statistics or higher mathematics).

FIGS. 5A and 5B show an exemplary patient risk score 38 as it might appear on the graphical user interface 30. The Patient Risk Score card shows the current outputs of various Early Warning Systems employed by the data analytics system 40 and the overall system 1 to drive the changes in the Model Parameters 52 and Dongle Function. Model Parameters 52 refer to the additional time scale synchronization features or changes to different EWS' frequencies that may be more applicable to a higher risk patient.

As discussed below, the Patient Risk Score 38 also impacts the functioning of each of the patient's medical instrument dongles 10. The higher the patient risk score, the higher the frequency of the data aggregation to the attached medical instruments 18. Patient risk scores 38 also may refer to a variety of standardized or proprietary methods of AE anticipation. For example, patient risk scores can utilize the standardized Modified Early Warning Scores (MEWS) as a means of increasing the data frequency; a patient with a high MEWS will have a higher frequency measurement than a patient who has a low MEWS.

In exemplary embodiments, the patient risk score is continuously calculated on the system's cloud platform and alerts practitioners of potential physiological decline. The higher the risk score, the more consistent a patient's metrics are trending with established AE precursors. The system alerts the attending practitioners when the patient risk score exceeds a certain threshold. More particularly, when one or more of the metrics generate a high patient risk score, the system 1 increases its frequency of measurement from a default state of low or medium frequency to medium or high frequency.

Thus, the dongle 10 or communication device 11 will ping the medical instrument 18 at a second, higher frequency. Therefore, patients with a high patient risk score will have their medical instruments polled at a higher frequency to provide attending nurses with higher resolution of data. The system 1 may be driven by software commands to increase the polling rate and frequency measurements. However, the system 1 can also take the form of hardware changes through changes in component voltages and crystal-based frequencies to effect these changes.

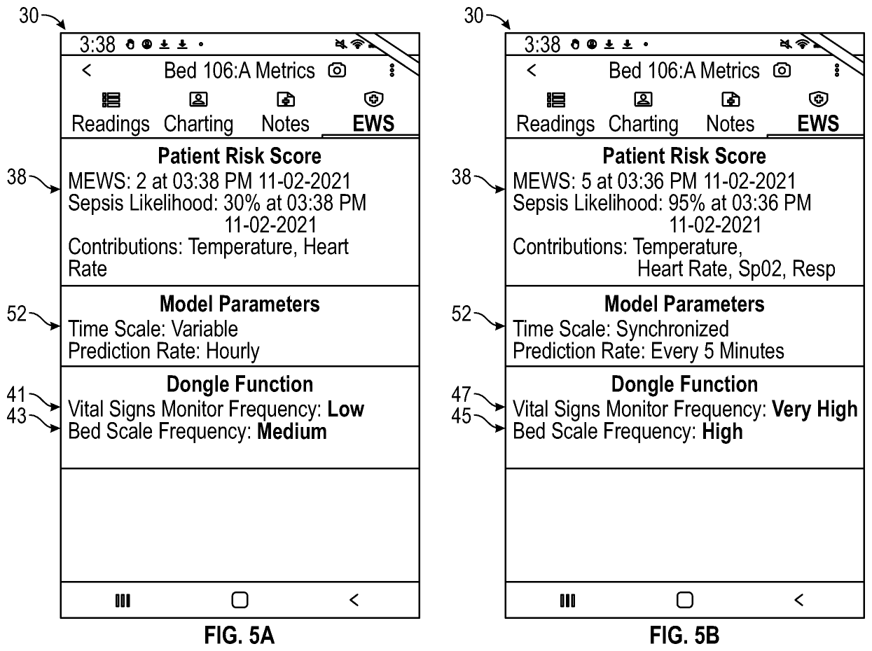

Turning to FIG. 6, the patient risk score 38 may also be used by the system 1 to normalize frequencies, i.e., normalizing asynchronous metric timescales for multiple measured metrics to improve adverse event anticipation. For example, pulse oxygen may be measured as a continuous waveform while heart rate may be measured every few seconds. In most cases, blood pressure and temperature are not taken frequently because the sensors are not connected to the patient. When a patient is connected to these sensors, a nurse must come in to manually take the vital signs. However, disclosed dongles 10 and systems (e.g., vital signs watch implemented by the system), can ping the medical instruments 18 and force readings at regular intervals which would satisfy the synchronous/asynchronous readings for multiple metrics.

In exemplary embodiments, if the patient risk score 38 exceeds its threshold value but does not exceed any boundary condition, the system 1 is set to medium and normalizes asynchronous timescales. If the patient risk score exceeds its threshold value and exceeds a single boundary condition, the system may be set to high and normalizes asynchronous timescales. In exemplary embodiments, if the patient risk score exceeds its threshold value and exceeds multiple boundary conditions, the system 1 is set to very high 47 and normalizes asynchronous timescales. Some values are not taken regularly, and when the dongle 10 receives one of these values, it will force ping the system 1 to get an accompanying data set for the regular metrics.

At the start, the dongle 10 or communication device 11 is set 510 to the standard data aggregation rate. It receives 520 a data packet from a medical instrument 18 and pushes 530 the data packet to the communication device 11. The dongle 10 then receives 540 a patient risk score 38 from the data analytics system 40 and the system 1 queries 550 whether the risk score is out of bounds. If it is, the system 1 queries 1040 whether the patient risk score 38 is above the low-risk threshold. If so, the dongle 10 sends 560 an interrupt to the aggregation routine and is set 570 to a faster data aggregation rate (e.g., medium or high frequency), increasing the frequency of its signals to the medical instrument from standard to medium or high. If the risk score is below the low-risk threshold, the dongle also sends 560 an interrupt to the aggregation routine and is set 570 to the fastest data aggregation rate (e.g., high or very high frequency), increasing the frequency of its signals to the medical instrument from medium to high or very high.

At this point, when the dongle 10 or communication device 11 receives 520 a data packet from the medical instrument 18 and pushes 530 it to the communication device 11, the system 1 queries whether any of the aggregated metrics are asynchronous. If the answer is in the affirmative, the dongle 10 or communication device 11 waits 590 for the asynchronous value to be measured and may send 600 a signal to the medical instrument for an additional reading, pushing 530 the additional data packet to the communication device 11.

In either case of AE potential, the system 1 will also switch to a higher frequency AE anticipation system. In most cases, outputs are hourly from the early warning system, and in the event of patients who exhibit high AE potential, the system will automatically switch to a higher frequency early warning system to consistently check on the patient's physiology and potential for decline.

Advantageously, the dongle 10 or communication device 11 is configured to switch back and forth between signaling synchronous (regular rate) monitoring or reading by the medical instruments 18 and asynchronous (irregular, triggered by an event or on demand of a medical practitioner) monitoring or reading as often as needed. Either in response to a concerning patient risk score or a request from a medical practitioner, the dongle 10 or communication device 11 can change from regular readings, signaled via its synchronous I/O unit 21, to asynchronous readings by routing through its asynchronous I/O unit 23.

This could be done when something tells the system there is a problem with a patient, e.g., a metric outside a boundary parameter or a concerning patient risk score, an infusion bag is empty, or on demand from a medical practitioner who has a reason for concern. The medical practitioner could tell the system via the user interface on her mobile device to request that, e.g., a vital signs monitor immediately take another reading. This would prompt the dongle 10 or communication device 11 to switch to its asynchronous I/O 23 and cause the vital signs monitor to interrupt its regular routine and take another reading immediately. If additional readings are no longer needed, the dongle 10 or communication device 11 can switch to signaling via its synchronous I/O unit 21 to change the vital signs monitor back to synchronous monitoring. Typically, the signal would be the same; the difference would be whether it is routed through the synchronous I/O unit 21 or the asynchronous I/O unit 23.

These switches can be done as many times as needed to provide the medical practitioner with the information he or she needs to effectively monitor patients. This flexibility and ability to switch quickly and fluidly is advantageous because a medical practitioner often does not know when they will receive a piece of medical data that requires immediate reaction. Because the user interface is on the medical practitioner's mobile device, synchronous processes can be made asynchronous and vice versa remotely.

An exemplary graphical or user interface 30 displays the history of the patient's progression based on a single metric, which could be the pulse. As it pertains to the patient proxy, the user/graphical interface 30 gives practitioners the ability to see trends in the data, as well as artifacts. The graphical interface 30 gives the practitioner the ability to see if the reason a metric rose above or fell below the threshold was a “sudden spike” (which typically refers to a malfunction) or an actual trend in which the patient may be suffering from an adverse event.

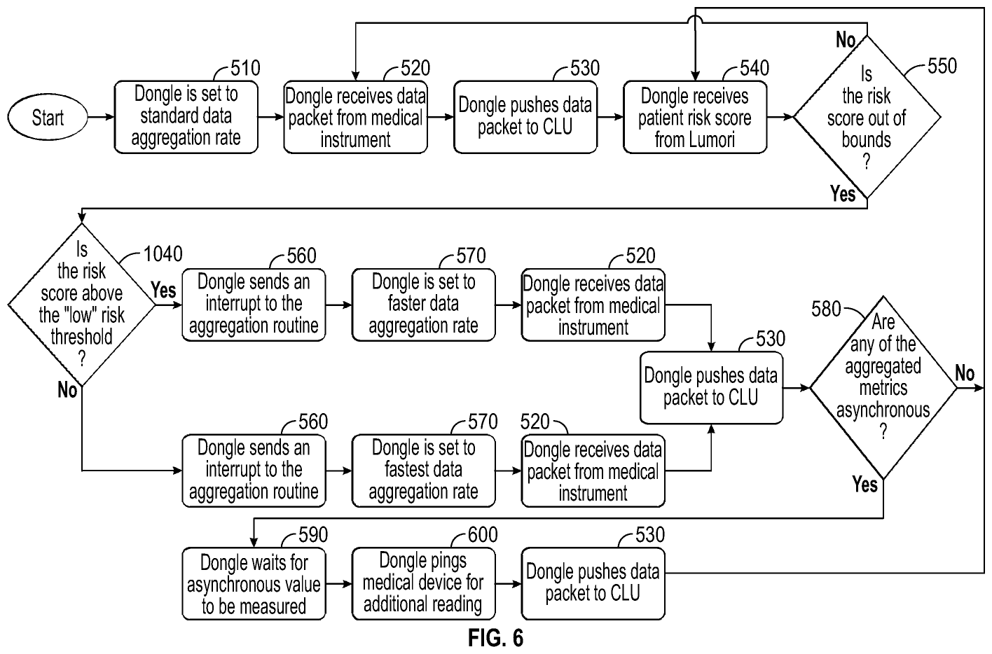

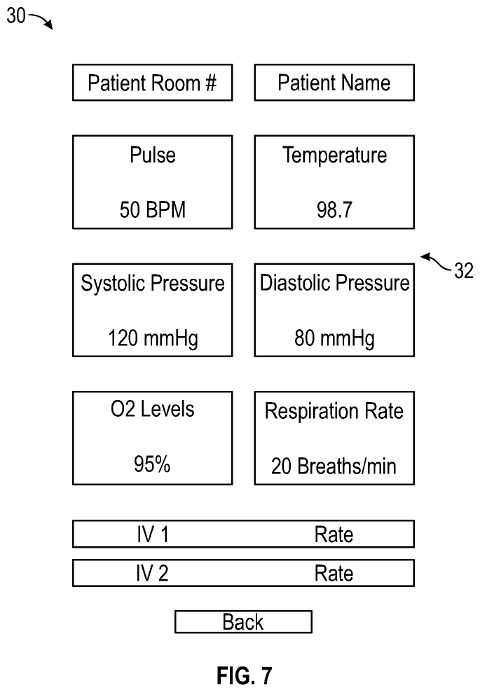

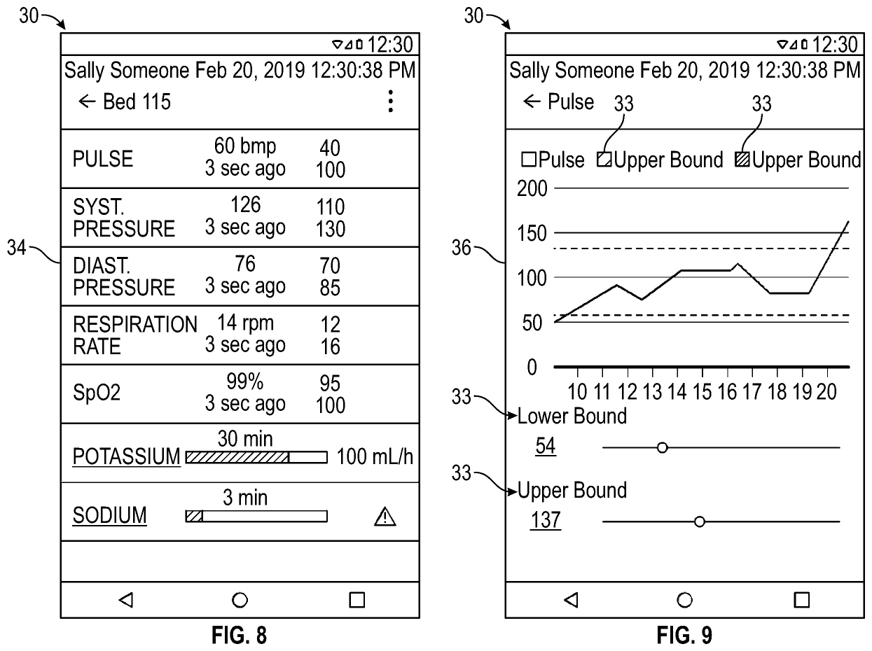

The user interface 30 typically has a home screen 32 for the mobile application, as shown in FIGS. 3 and 7. The purpose of this screen is to give the healthcare practitioner a quick method to ascertain the status of his or her patients; the first “home” page of a patient proxy is a way for practitioners to quickly see how their patient is doing. With reference to FIG. 8, an exemplary in-depth page 34 might appear when a medical practitioner selects any of the rooms or beds displayed on a home screen 32. Here, a practitioner would be able to see how all the medical instruments are operating, enter various boundary conditions, and ensure that the patient is within those conditions. An example of a patient's progression provided by graphing screen 36 is illustrated in FIG. 9. Each metric shown on the application is measured from the treatment and medical instruments connected to the patient (in this case Infusion Pumps, Vital Signs Monitors, and Bed Scales) and may be transferred to a mobile interface via the communication device 10 and/or the cloud.

In exemplary embodiments, for each metric there are numbers on the right that act as soft boundaries for the metric measured. Exemplary embodiments may have a screen in which the user can view all the alarms that have sounded, either from the medical instrument or mobile device itself, sortable by each metric. This may be implemented on a device level and/or may be viewable on an EMR level.

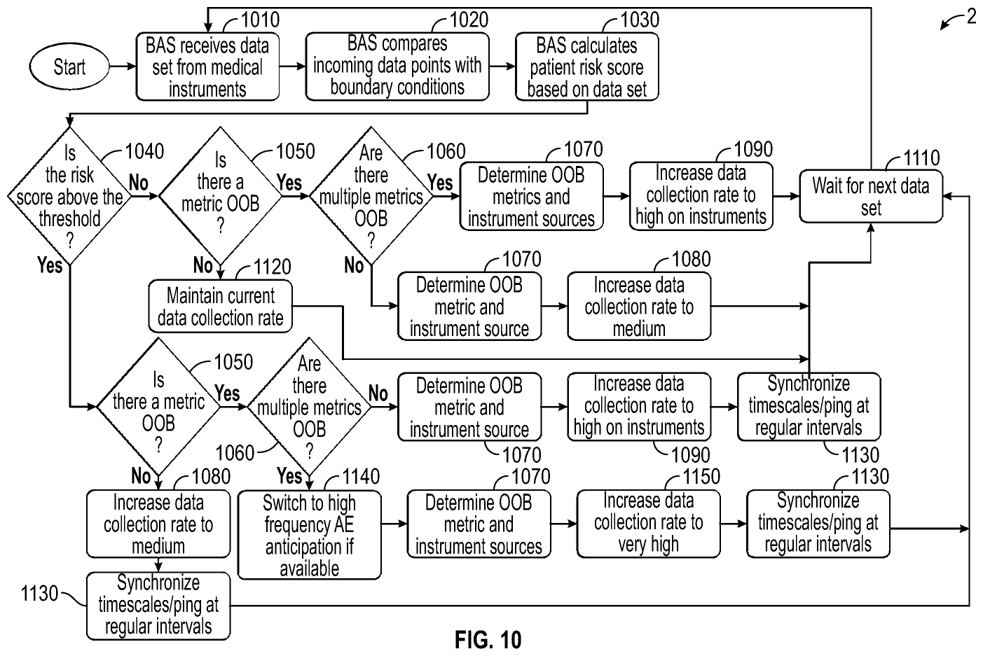

In operation, the dongles 10 and/or communication device 11 are attached to and/or communicatively coupled to the medical instruments 18 to be used for monitoring patients. Each dongle 10 begins to send periodic signals to its respective medical instrument 18 at the pre-set baseline or default frequency. Via the user interface 30 on a mobile device 12, a nurse or other medical practitioner may set boundary conditions for the metrics measured by the medical instruments 18 as a function of the early warning system 22. Referring to FIG. 10, in exemplary methods 2 the data analytics system 40 receives 1010 data sets from the medical instruments 18 and compares 1020 the incoming data points with the boundary conditions set by the medical practitioner.

Using the incoming data points, the data analytics system 40 may calculate 1030 a patient risk score 38 based on the data sets. Then the data analytics system 40 queries 1040 whether the patient risk score 38 is above the pre-determined threshold. As part of the patient risk score determination or as separate inquiries, queries are made whether there is one metric out of bounds (1050) and/or multiple metrics out of bounds (1060). If there is no metric exceeding it boundary parameter, the current data collection rate is maintained 1120.

If there is one or multiple out of bounds metrics, the next step is to determine 1070 the out of bounds metric(s) and the corresponding medical instrument source(s). If one metric exceeds its boundary parameter, the dongle 10 pings the medical instrument 18 at a higher frequency, raising the measurement frequency from low to medium. If more than one metric exceeds its boundary parameter, the dongle 10 pings the medical instrument 18 at a high frequency, raising the measurement frequency from low or medium to high. Thus, the rate of data collection is raised 1080 to medium or raised 1090 to high and the system 1 waits 1110 for the next data set.

If the patient risk score 38 is above the threshold, the system 1 synchronizes 1130 timescales at regular intervals for one or more measured metrics. If multiple measured metrics are out of bounds, the system 1 also may switch 1140 to higher frequency AE anticipation if available. This might include increasing 1150 the data collection rate to very high 47 accompanied by synchronization 1130 of timescales at regular intervals. With a determination that one or more metrics exceed their boundary parameters, alarms 35 would be sent to the medical practitioner to alert him or her of the changes in medical conditions of the patient.

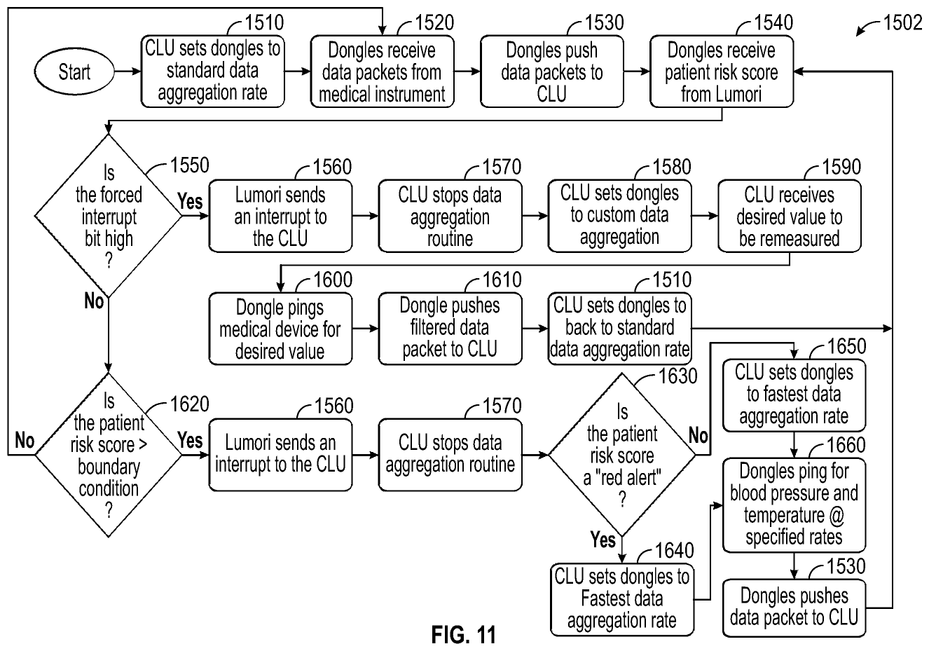

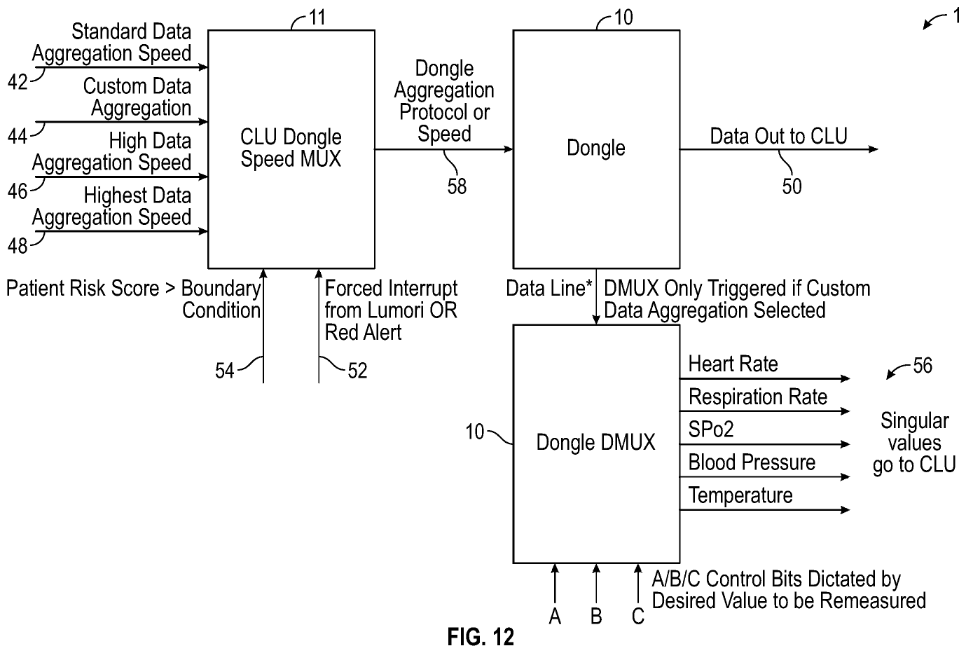

With reference to FIGS. 11 and 12, an exemplary method 1502 of switching between synchronous and asynchronous monitoring including pushing different data frequencies by adjusting the dongle aggregation speed 58 is shown. FIG. 12 is an example diagram of a system 1 using the MUX/DMUXs to push different data frequencies. At the start, the communication device 11 sets 1510 the dongles 10 to the standard data aggregation rate 42. The dongles 10 receive 1520 data packets from the medical instruments 18 and push 1530 the data packets 50 to the communication device 11. Then the dongles 10 receive 1540 a patient risk score 38 from the system 1.

The system 1 queries 1550 whether the forced interrupt 52 bit is high and, if so, the system 1 sends 1560 an interrupt to the communication device 11, which stops 1570 the data aggregation routine. The communication device 11 then may set 1580 the dongles 10 to a custom data aggregation rate 44. Once the communication device 11 receives 1590 the desired value to be measured, the dongle 10 pings 1600 the medical device 18 for the desired value. The dongle 10 then pushes 1610 the filtered data packet to the communication device 11, which sets 1510 the dongles 10 back to the standard data aggregation rate 42.

If the forced interrupt bit is not high, the system 1 queries 1620 whether the patient risk score 38 is out of the range 54 of a boundary condition. If so, the system sends 1560 an interrupt to the communication device 11, which stops 1570 the data aggregation routine. The next query 1630 is whether the patient risk score is a red alert. If so, the communication device 11 sets 1640 the dongles 10 to the highest or fastest data aggregation rate 48. If not, the communication device 11 sets 1650 the dongles to a high or fast data aggregation rate 46. In either case, the dongles 10 then ping 1660 for vital signs readings 56 such as blood pressure, respiration rate, and temperature at specified rates and push the data packets to the communication device 11.

Thus, it is seen that devices, systems, and methods for controlling and varying medical instrument monitoring of patients and for switching between synchronous and asynchronous monitoring are provided. It should be understood that any of the foregoing configurations and specialized components may be interchangeably used with any of the systems of the preceding embodiments. Although illustrative embodiments are described hereinabove, it will be evident to one skilled in the art that various changes and modifications may be made therein without departing from the disclosure. It is intended in the appended claims to cover all such changes and modifications that fall within the true spirit and scope of the disclosure.

While the disclosed systems and devices have been described in terms of what are presently considered to be the most practical exemplary embodiments, it is to be understood that the disclosure need not be limited to the disclosed embodiments. It is intended to cover various modifications and similar arrangements included within the spirit and scope of the claims, the scope of which should be accorded the broadest interpretation so as to encompass all such modifications and similar structures. The present disclosure includes any and all embodiments of the following claims.

1. A dongle for controlling and varying medical instrument monitoring of patients, comprising:
a microcontroller;
an antenna in communication with the microcontroller;
the dongle being configured to receive data from a medical instrument, the data including one more metrics being measured by the medical instrument, the dongle being configured to send periodic signals of a first frequency to the medical instrument;
wherein when one or more of the one or more metrics exceeds a pre-determined boundary parameter or one or more aggregated metrics generate a high patient risk score, the dongle sends periodic signals of a second frequency to the medical instrument, the second frequency being higher than the first frequency.

2. The dongle of claim 1 wherein when a single metric exceeds the pre-determined boundary parameter the second frequency is a medium frequency and when more than one metric exceeds the pre-determined boundary parameter the second frequency is a high frequency.

3. The dongle of claim 3 wherein when more than one metric exceeds the pre-determined boundary parameter the dongle prompts the medical instrument to manually measure the more than one metric at specified time intervals.

4. The dongle of claim 1 wherein changes in frequency of the signals sent by the dongle are enabled by hardware changes comprising one or more of: changes in component voltages and changes in crystal-based frequencies.

5. The dongle of claim 1 wherein the microcontroller compares the measured metrics to the pre-determined boundary parameters.

6. The dongle of claim 1 further comprising an IUI connector.

7. The dongle of claim 1 wherein the microcontroller analyzes aggregated measured metrics for adverse event anticipation and to generate the patient risk score and uses the patient risk score to normalize asynchronous specified time intervals for more than one measured metric.

8. A communication system for controlling and varying medical instrument monitoring of patients, comprising:
a dongle including a microcontroller and an antenna in communication with the microcontroller;
a data analytics system including a computer-readable storage medium and implementing instructions stored in the computer-readable storage medium;
the dongle being configured to receive data from a medical instrument, the data including one more metrics being measured by the medical instrument, the dongle being configured to send periodic signals of a first frequency to the medical instrument;
wherein when one or more of the one or more metrics exceeds a pre-determined boundary parameter or one or more aggregated metrics generate a high patient risk score, the dongle sends periodic signals of a second frequency to the medical instrument, the second frequency being higher than the first frequency.

9. The communication system of claim 8 wherein when a single metric exceeds the pre-determined boundary parameter the second frequency is a medium frequency, and when more than one metric exceeds the pre-determined boundary parameter the second frequency is a high frequency.

10. The communication system of claim 9 wherein when more than one metric exceeds the pre-determined boundary parameter the dongle prompts the medical instrument to manually measure the more than one metric at specified time intervals.

11. The communication system of claim 8 wherein the data analytics system analyzes aggregated measured metrics for adverse event anticipation to generate the patient risk score and uses the patient risk score to normalize asynchronous specified time intervals for more than one measured metric.

12. The communication system of claim 8 further comprising an early warning system including a user interface and an alarm, the communication system communicating information about a patient's medical condition based on the data from the medical instrument to a mobile device in real time.

13. The communication system of claim 12 wherein the medical instrument is one or more of an infusion pump, a vital signs monitor, and a bed scale.

14. The communication system of claim 12 wherein the data analytics system determines whether any of the data from the medical instrument is outside the pre-determined boundary parameter and determines likelihoods that a patient may experience an adverse event through comparisons between the data from the medical instrument and data in an adverse event repository.

15. The communication system of claim 14 wherein the alarm alerts the medical practitioner when stored data is outside the pre-determined boundary parameter, or the at least one medical instrument has stopped functioning or changed its rate, or a patient may experience an adverse event.

16. The communication system of claim 13 wherein the medical instrument is an infusion pump, and the alarm alerts a medical practitioner when pressure builds on an IV line connected to the infusion pump.

17. The communication system of claim 13 wherein the medical instrument is a bed scale including a load sensor, and the dongle collects load scale data from the load sensor.

18. A communication system for switching between synchronous and asynchronous monitoring of a patient, comprising:
a dongle including a microcontroller;
a communication device in communication with the dongle;
a data analytics system including a computer-readable storage medium and implementing instructions stored in the computer-readable storage medium; and
a synchronous input/output unit and an asynchronous input/output unit;
the dongle being configured to receive data from a medical instrument, the data including one more metrics being measured by the medical instrument;
the communication system being configured to route a signal via the synchronous input/output unit to the medical instrument to cause the medical instrument to provide regular readings of the data and to route a signal via the asynchronous input/output unit to the medical instrument to cause the medical instrument to provide additional or irregular readings of the data and to switch between the synchronous input/output unit and the asynchronous input/output unit on demand or based on prior instructions from a user.

19. The communication system of claim 18 wherein the dongle has the synchronous input/output unit and the asynchronous input/output unit.

20. The communication system of claim 18 wherein the communication device has the synchronous input/output unit and the asynchronous input/output unit.
